(19) United States
(12) Patent Application Publication
(10) Pub. No.: US 2013/0230911 A1
(43) Pub. Date: Sep. 5, 2013

(54) POROUS STRUCTURE WITH INDEPENDENTLY CONTROLLED SURFACE PATTERNS

(71) Applicant: THE CHARLES STARK DRAPER LABORATORY, INC., Cambridge, MA (US)

(72) Inventors: Joseph L. Charest, Cambridge, MA (US); Jeffrey T. Borenstein, Newton, MA (US); Else Forhlich, Brookline, MA (US)

(73) Assignee: THE CHARLES STARK DRAPER LABORATORY, INC., Cambridge, MA (US)

(21) Appl. No.: 13/784,271

(22) Filed: Mar. 4, 2013

Related U.S. Application Data
(60) Provisional application No. 61/606,087, filed on Mar. 2, 2012.

(51) Int. Cl. C12M 1/12; B29C 59/00

(52) U.S. Cl. CPC C12M 25/02; B29C 59/00
USPC 435/288.7; 435/297.1; 435/287.1; 264/48

(57) ABSTRACT

Disclosed herein are systems and methods for manufacturing and using a cell culture support device. The device includes a plurality of polymer layers, each with at least one flow chamber defined therethrough. The device also includes a cross channel interface between the channels of different polymer layers. The cross channel interface includes a plurality of pores and a topographical pattern that is selected independent of the plurality of pores. Furthermore, the formation of the topographical pattern preservers the pores.

### CROSS-REFERENCE TO RELATED PATENT APPLICATIONS

This application claims priority to Provisional U.S. Patent Application 61/606,087, filed Mar. 2, 2012, which is incorporated herein by reference in its entirety.

### BACKGROUND OF THE DISCLOSURE

Reabsorptive transport in vivo occurs through natural barriers, formed by a single layer of polarized epithelial cells supported by a basement membrane (BM) which governs the transport. Solutes and molecules cross the epithelial barrier by transcellular or paracellular pathways to the interstitial space and surrounding blood vessels, resulting in reabsorption of essential water and solutes. Common examples of reabsorptive or absorptive barriers in the body include those of the respiratory, gastrointestinal, and urinary tracts. Fluid and solute transport across these barriers make them particularly susceptible to injury by circulating toxins, pathogenic antibodies or certain drugs.

### SUMMARY OF THE DISCLOSURE

According to one aspect of the disclosure, a cell culture support device includes a first and second polymer layer, each with a flow chamber defined therethrough. Additionally, the cell culture support device includes a surface (also referred to as a cross channel interface) between the first polymer layer and the second polymer layer. The surface separates the first flow chamber from the second flow chamber. The surface includes a plurality of pores configured to allow communication and transport between the flow chambers. Furthermore, the surface includes a pattern independent of the geometry of the plurality of pores.

In some implementations, the surface is a membrane and the flow chambers are configured to be cellular chambers. In some implementations, the top layer of the device is configured to allow imaging of the surface in some implementations.

In certain implementations, the pattern is a topographic pattern and/or a chemical pattern. The pattern can be selected to enhance the growth of specific cell types. In some implementations, more than one pattern is formed on the face of the surface and/or flow chambers.

In other implementations, the pores are selected to produce a specific type of interaction between the flow chambers. In some implementations, the size of the pores is selected to prevent cell migration between the flow chambers but to allow cell nutrients and cell signaling analytes to migrate between the flow chambers. The size of the pores is between about 3 μm and about 15 μm in some implementations. In other implementations, the pattern and/or the geometry of the pores is selected to elicit a particular arrangement, function, shape, or density of cellular growth.

In yet other implementations, at least one of the polymer layers and/or the surface includes a biodegradable polymer. In some implementations, the pattern is selected to influence a degradation rate of the surface and/or to facilitate cellular attachment to particular locations within the cell culture support device.

According to another aspect of the disclosure, a method for fabricating a cell culture support device includes forming a first flow chamber in a first polymer layer, and forming a second flow chamber in a second polymer layer. Additionally, pores of a specific size are formed through a surface. A selected pattern is then applied to the surface. The selection of the pattern is independent from the selection of the pore size and position. The forming is done such that the formation of the pattern on the surface preserves the plurality of pores. Additionally, the method includes coupling the first polymer layer and the second polymer layer such that the surface separates the first flow chamber from the second flow chamber.

In some implementations, cells are seeded into at least one of the flow chambers. In certain implementations, the pores are formed such that they have a specific pore density along the surface. In other implementations, the pattern is a topographic pattern and/or a chemical pattern selected responsive to the type of cells to be grown on the surface.

In some implementations, the method also includes selecting and forming additional patterns onto the surface and or walls of the flow chamber. The additional patterns can be the same as, or different than, the initially selected pattern. In some implementations, the patterns are selected to elicit a particular arrangement, function, shape, or density of cells grown on the surface.

In yet other implementations, the polymer layers and/or surface include a biodegradable polymer. The pattern is selected to influence a degradation rate of the surface in some implementations.

According to yet another aspect of the disclosure, a cell culture support system includes a first and second polymer layer each with flow chambers defined therethrough and a surface separating the flow chamber of the first polymer layer from the flow chamber of the second polymer layer. The surface includes a plurality of pores configured to allow communication and transport between the flow chambers. Additionally, at least one face of the surface is patterned. The pattern is independent of the geometry of the pores and preserves the size of the pores when formed. The system also includes an imager configured to image a face of the surface.

In certain implementations, the system includes a means for coupling the surface between the first and second polymer layers, a flow meter configured to measure flow through at least one of the flow chambers, a pressure sensor configured to measure the pressure at an inlet and/or an outlet of the flow chambers, a fluid pump configured to flow fluid through the flow chambers, and macro-molecule injector coupled to an inlet of the flow chambers.

### BRIEF DESCRIPTION OF THE DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 is a block diagram of an example system in which a cell culture support device is employed.

FIG. 2 is a solid model of an example cell culture support device, as can be employed in the system of FIG. 1.

FIG. 3 is a cross sectional view of an example cell culture support device, in which the flow chambers are seeded with cells.

FIGS. 4A-4C are a series of solid models illustrating example cross channel interface pore topographies.

FIG. 5 is a flow chart of an example method for manufacturing a cell culture support device similar to the device of FIG. 2.

FIG. 6 is a flow chart of an example method for using a cell culture support device in a system similar to the system of FIG. 1.

FIG. 7A is a cross sectional schematic of an example cell culture support device.

FIG. 7B is an image of a cell culture support device manufactured based on the schematic shown in FIG. 7A.

FIGS. 7C-7E are a series of scanning electron micrographs, at various magnifications, of the cell culture support device shown in FIG. 7B.

FIGS. 8A-8E are a series of scanning electron micrographs showing topographies of various example cross channel interfaces.

FIG. 9A is a plot illustrating the relationship between the change in pore diameter and hot-embossing dwell time.

FIG. 9B is a plot illustrating how pore diameter is affected by cross channel interface topography when hot embossed.

FIG. 9C is a series of scanning electron micrographs of example cross channel interfaces with various topographies.

FIG. 9D is a plot illustrating how pore elongation is affected by cross channel interface topography and pore diameter.

FIG. 10A is a brightfield microscopy image of a cell culture support device's flow chamber seeded with HK-2 cells.

FIG. 10B is a brightfield microscopy image of the same flow chamber show in FIG. 10A, viewed under higher magnification.

FIG. 10C is a series of confocal microscopy images of the cells seeded in the flow chamber shown in FIG. 10A.

FIG. 10D is a brightfield microscopy image of a cell culture support device's flow chamber seeded with primary renal proximal tubule epithelial cells.

FIG. 10E is a brightfield microscopy image of the same flow chamber shown in FIG. 10D viewed under higher magnification.

FIG. 10F is a series of confocal microscopy images of the cells seeded in the flow chamber shown in FIG. 10D.

FIG. 11A is a scanning electron micrograph of an example cross channel interface prior to cellular seeding.

FIG. 11B is a scanning electron micrograph of an example cross channel interface after a cellular mat has formed on the cross channel interface.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

Models of absorptive barriers in the respiratory, gastrointestinal, and urinary tracts would offer a platform to better understand the biology and function of reabsorptive barriers, to interrogate underlying disease mechanisms affecting those barriers, and to provide rapid screening of drugs for toxic effects to and excretion by organs containing those barriers. In particular, since the kidney is susceptible to drug toxicity and governs excretion of drugs, its renal epithelial structures provide valuable test cases for in vitro models of reabsorptive barriers. Disclosed herein is a systems and methods for the manufacture and use of such barriers in vitro. In some implementations, the systems and methods disclosed herein are used as a medical device to assist organ function.

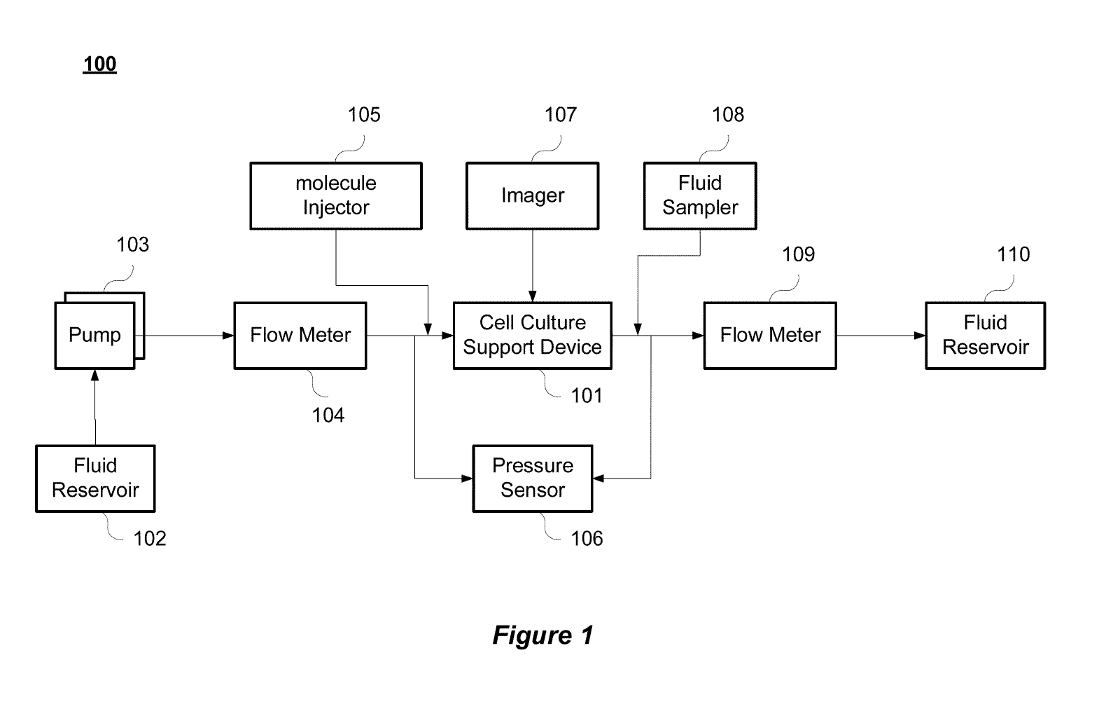

FIG. 1 illustrates a cell culture support system 100. The system 100 includes at least one cell culture support device 101. The system 100 also includes at least one pump 103 that pumps fluid from a first fluid reservoir 102 into an inlet of the cell culture support device 101. As the fluid passes from the pump 103 to the cell culture support device 101, it passes through a flow meter 104 and past a molecule injector 105. Upon exiting the cell culture support device 101 the fluid passes by a fluid sampler 108 and through a second flow meter 109 and is then deposited into a second fluid reservoir 110. A pressure sensor 106 measures the pressure at, or near, the inlet and the outlet of the cell culture support device 101. Additionally, the system 100 includes an imager 107, which is used to view cells within the cell culture support device 101.

As discussed above, the system 100 includes a number of components to support the cell culture support device 101. The pump 103 drives fluid from the first fluid reservoir 102 through the cell culture support device 101. In some implementations, the pump 103 is a peristaltic pump or a syringe pump. In implementations using a syringe pump, the fluid reservoir 102 is the barrel of a syringe. The pump 103 controls the fluid flowing through the cell culture support device 101. For example, the pump can control the fluid's flow rate and the duration of the flow through the cell culture support device 101. In some implementations, the flow is continuous and in other implementations the flow is pulsatile. The pump 103 can be configured to control the shear stress the fluid exerts on cells within the cell culture support device 101. The fluids passed through the cell culture support device 101 can include, but are not limited to, cell culture medium, cell nutrients, reagents, test agents, buffer fluids, reactant fluids, fixing agents, stains, simulated and real biological fluids such as blood filtrate, whole blood, blood serum, blood plasma, urine, dilute urine.

In some implementations, the above agents and/or other molecules are added to the fluid flowing into the cell culture support device 101 by the molecule injector 105. In certain implementations, the molecule injector 105 is a second syringe pump. In other implementations, continuous delivery of nutrients by the fluid creates favorable conditions for long term cell culture within the cell culture support device 101. The system 100 also includes a fluid sampler 108. The fluid sampler 108 is positioned near the outlet of the cell culture support device 101. In some implementations, the fluid sampler 108 is configured to siphon off a small amount of the fluid exiting the cell culture support device 101. The collected fluid may be tested for specific molecular markers, reagents, or other such molecules.

The system 100 further includes a flow meter 104 near the inlet of the cell culture support device 101, a flow meter 109 near the outlet of the cell culture support device 101, and a pressure sensor 106. The pressure sensor 106 measures the pressure of the fluid as it enters and exits the cell culture support device 101. In certain implementations, the measurements made by the flow meters 104 and 109 and the pressure sensor 106 are used to calculate the shear stress imparted on cells within the cell culture support device 101.

The imager 107 is used to observe cells within the cell culture support device 101. In some implementations, the cells are imaged while fluid is flowing through the cell culture support device 101. In other implementations, at the end of an experiment fixing fluid is passed through the cell culture support device 101 and the cells are imaged upon completion of experimentation. In other implementations, the imager 107 is configured to monitor the integrity of the cross channel interface. For example, the imager 107 can be configured to measure the degree to which the cross channel interface 202 has degraded.

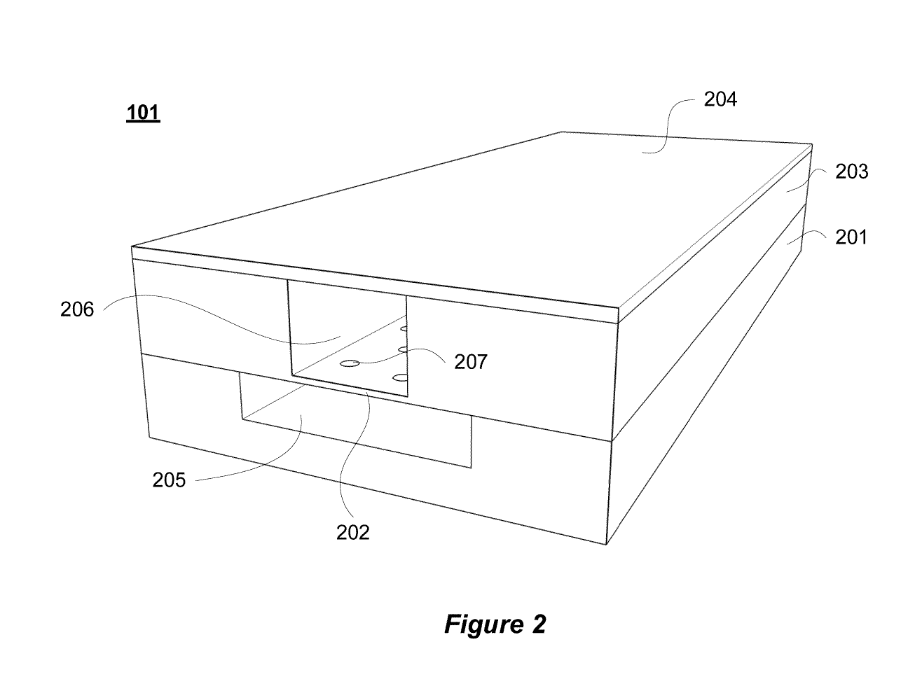

FIG. 2 is a solid model illustrating the cell culture support device 101 in greater detail. As illustrated, the cell culture support device 101 is a multi-layered polymer device. The cell culture support device 101 includes a first polymer layer 203 with a first flow chamber 206 defined therethrough, and a second polymer layer 201 has a second flow chamber 205 defined therethrough. The first polymer layer 203 of the cell culture support device 101 also includes a cross channel interface 202. In some other implementations, the cross channel interface 202 is an additional polymer layer that is coupled between the first polymer layer 203 and second polymer layer 201. In certain implementations, the cross channel interface 202 is a membrane made of a thermoplastic, such as polystyrene, polycarbonate, polyimide, polysulfone, polyethersulfone; biodegradable polyesters, such as polycaprolactone (PCL); soft elastomers, such as polyglycerol sebacate (PGS); or other polymers such as polydimethylsiloxane (PDMS) and poly(N-isopropylacrylamide). In yet other implementations, the cross channel interface 202 is made from silicon, glass, or silicon nitride. The cross channel interface 202 is manufactured, in some implementations, through processing methods such as track-etching, electro-spinning, microfabrication, micromolding, gel deposition, phase separation, particle leaching, and solvent leaching. In yet other implementations, the cross channel interface 202 is a multilayered membrane that includes several layers of material. For example, the material can be a structural backing, a skin layer, a porous layer, a layer that serves as a permeable spacer, or allows lateral flow.

As discussed above, in certain implementations, the interior of the cell culture support device 101 is imaged with the imager 107. Accordingly, in some implementations, the roof 204 of the first flow chamber 206 is configured to allow for visual inspection of the first flow chamber 206, cross channel interface 202, and/or second flow chamber 205. In other implementations, the roof 204 is a polymer layer manufactured out of a material similar to, or the same as, the polymer layers. In certain implementations, the cell culture support device 101 includes more than one flow chamber within a polymer layer. Additionally, in some implementations, the cell culture support device 101 includes more than two polymer layers with flow chambers. For example, the cell culture support device 101 can include three or more polymer layers each separated from one another by a different cross channel interface 202. The polymer layers can include, but are not limited to, a thermoplastic, such as polystyrene, polycarbonate, polyimide; biodegradable polyesters, such as polycaprolactone (PCL); soft elastomers, such as polyglycerol sebacate (PGS); or other polymers such as polydimethylsiloxane (PDMS) and poly(N-isopropylacrylamide). In certain implementations, the polymer material is selected for its ability to be micro-machined and support cell growth. In some implementations, the length, width, and height of the flow chambers are selected to mimic kidney structures. In other implementations, the height of a flow chamber is between about 10 μm and about 100 μm, the width is between about 250 μm and about 2 mm, and the length is between about 5 mm and about 10 mm.

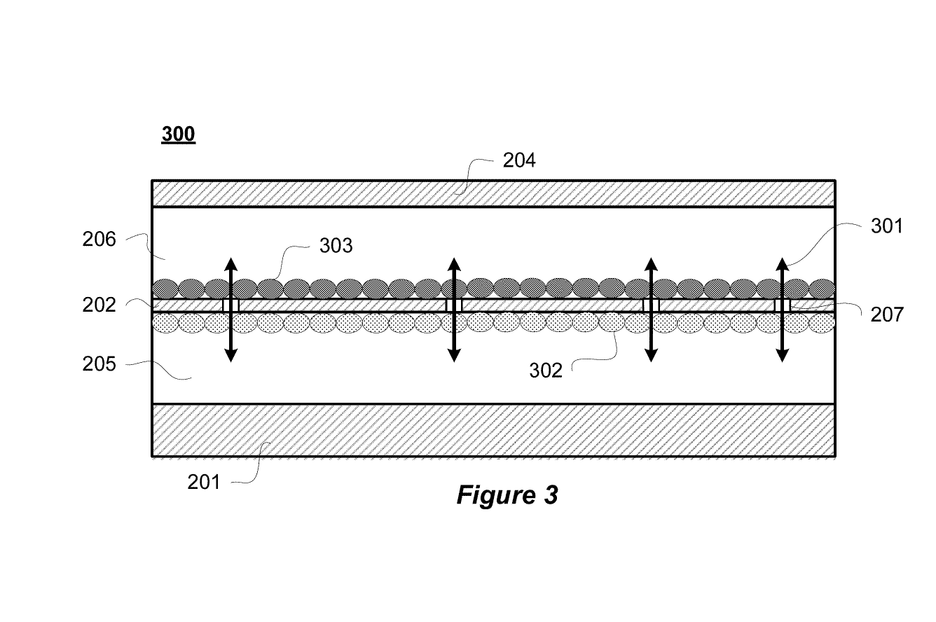

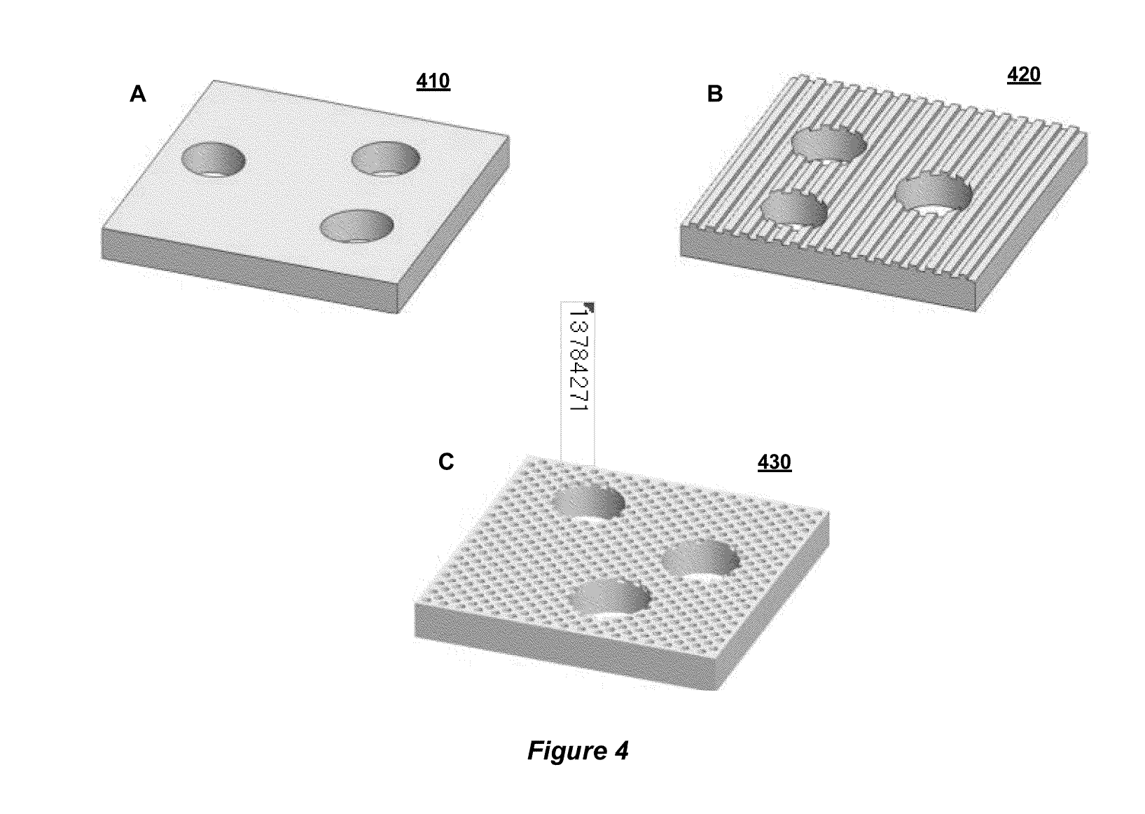

Discussed in greater detail in relation to FIGS. 3 and 4A-4C, but briefly, the cross channel interface 202 enables communication between the first flow chamber 206 and the second flow chamber 205. A plurality of pores 207 are defined through the cross channel interface 202 and at least one face of the cross channel interface 202 includes a topography that is independent of the pores 207. A cross channel interface 202 with pores 207 that are created independent of the topography generates a porous membrane that facilitates basement membrane (BM)-like architecture and enables better control of experimental variables. In some implementations, the topography is selected such that it has a specific effect on the pores 207. For example, the topography can be selected such that it alters the pore share or closes the pores in a specific area of the cross channel interface 202 or reduces the size of the pores 207 by a specific size.

In some implementations, the pores 207 of the cross channel interface 202 are generated by track-etching. Track-etching creates highly uniform pores 207. The pore sizes range between about 3 μm and 15 μm wide. The cross channel interface 202 is between about 6 μm and 30 μm thick.

As indicated above, at least one face of the cross channel interface 202 is patterned with a selected topography. In certain implementations, at least one wall of the first flow chamber 206 and/or second flow chamber 205 is also patterned with a selected topography. In some implementations, the patterned faces (e.g. a cross channel interface 202 face and a first flow chamber 206 wall) are patterned with a different topographies. In yet other implementations, different sections of the same face are patterned with different topographies.

FIG. 3 is a cross-sectional view of a cell culture support device 300 similar to the cell culture support device 101 of FIG. 2. The cell culture support device 300 includes a first polymer layer in which a first flow chamber 206 is defined, and a second polymer layer 201 in which a second flow chamber 205 is defined. A cross channel interface 202 forms the roof of the second flow chamber 205 and the floor of the first flow chamber 206. A top polymer layer 204 forms the roof of the first flow chamber 206. As previously discussed, the cross channel interface 202 includes a plurality of pores 207. As illustrated, the cell culture support device 300 includes a first plurality of cells 302 seeded in the first flow chamber 206 and a second plurality of cells 303 seeded in the second flow chamber 205. In some implementations, cells are only seeded into one of the flow chambers 206 and 205. As illustrated by the flow arrows 301, fluid communication occurs through the pores 207. The fluid communication can include, but is not limited to, the passive transport of macromolecules, nutrients, and test agents. In some implementations, the size of the pores 207 allows for cells to migrate across the cross channel interface 202 and in other implementations the size and/or shape of the pores 207 inhibits trans-chamber migration of cells but allows for the migration of nutrients and cellular signaling analytes between the chambers. The pores 207 can be dispersed randomly or in an ordered fashion throughout the length of the cross channel interface 202, and in some implementations, the pores 207 are limited to specific regions of the cross channel interface 202. In some implementations, the arrangement, shape, and size is referred to as the pore geometry.

FIG. 4A-4C are solid models illustrating topographical patterns that can be applied to a cross channel interface. For illustrative purposes the cross channel interfaces includes three pores 207; however, the is no requirement for similar pore spacing, alignment, or concentrations. The topography of a cross channel interface 202 is selected based on the sells that are to be seeded into the cell culture support device 101, and can be selected to effect a particular arrangement, function, shape and/or density of cells. In some implementations, the cross channel interface 202 is manufactured from a biodegradable polymer. In some of these implementations, the surface pattern is selected to facilitate and/or control the degradation of the cross channel interface 202. In certain implementations, the topographies are selected and manufactured such that they degrade at a specific rate or when exposed to a specific chemical agent. For example, the patterning topography and cross channel interface 202 may be configured such that the cross channel interface 202 completely dissolves once a cellular mat has grown on the cross channel interface 202. Thus, in such an implementation, after cross channel interface 202 degradation, the first polymer layer 203 and second polymer layer 201 are separated only by the cellular mat.

FIG. 4A illustrates a portion of cross channel interface 410 that is smooth and does not contain a topographical pattern. FIG. 4B illustrates a cross channel interface 420 with a ridge and groove pattern. The ridge and groove pattern can be aligned perpendicular to, parallel with, or angled to the flow of fluid through a flow chamber. In other implementations, the ridges are between about 0.5 μm and about 1.0 μm wide, have a pitch between about 1.0 μm and about 2 μm, and are between about 0.5 μm and 1.0 μm tall. In some implementations, the dimensions and spacing of the ridges is constant along the entirety of a patterned surface. In other implementations, one or more of the ridge parameters is varied along the length of the patterned surface. For example, the spacing between the ridges may start at 1.0 μm and transition to a 2.0 μm spacing at the flow chamber's outlet. In certain implementations, the ridges and/or grooves are rounded.

FIG. 4C illustrates a cross channel interface 430 with a pit pattern. As illustrated, the pits are cylinderical, but in other implementations the pits can be rectangular, square, frustroconical, conical, and/or hemispherical. Additionally, the above pit patterns can be inversed to create posts. The pits and posts can be patterned in a regular, ordered fashion or randomly. For example, in the regular, ordered fashion, hemispherical posts may be aligned in ordered rows with 1.0 μm between each post of a row and 1.5 μm between each row. In some implementations, the cross channel interface 202 topographies includes a plurality of the above described topographies.

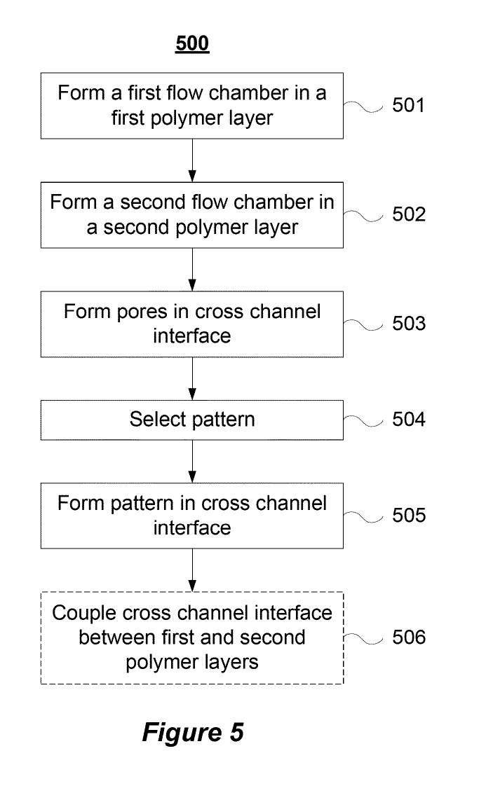

FIG. 5 is a flow chart of a method 500 for manufacturing a cell culture support device, such as that of system 100. The method 500 includes forming a first flow chamber in a first polymer layer (step 501), and also forming a second flow chamber in a second polymer layer (step 502). Additionally, the method 500 includes forming pores in a cross channel interface (step 503). A topographical pattern is selected (step 504) and formed into the cross channel interface (step 505). In some implementations, the cross channel interface is coupled between the first polymer layer and the second polymer layer (step 506).

As set forth above, a first flow chamber is manufactured in a first polymer layer (step 501) and a second flow chamber is manufactured in a second polymer layer (step 502). The flow chambers can be manufactured by photolithographic techniques, injection molding, direct micromachining, deep RIE etching, hot embossing, or any combinations thereof. In some implementations, as illustrated in FIG. 2, the cross channel interface 202 is a component of the first polymer layer 203. In other implementations, the cross channel interface 202 is a separate component that is separately manufactured and subsequently coupled between the first and second polymer layers.

The method 500 of manufacturing a cell culture support device continues with the formation of pores in the cross channel interface (step 503). In some implementations, the pores 207 of the cross channel interface 202 are manufactured by leaching micro-particles from the cross channel interface 202, phase separation micro-molding, track etching, or any combination thereof. In certain implementations, the cross channel interface 202 is obtained prefabricated with pores 207.

The method 500 continues with the selection of a topographical pattern to apply to the cross channel interface (step 504) and then the application of the selected pattern to the cross channel interface (step 505). As discussed above, the selection of the pattern can be dependent on the cells to be grown on the cross channel interface 202 and/or the desired arrangement, function, shape, and density of the seeded cells. Responsive to selecting the topographical pattern, the pattern is applied to the cross channel interface 202.

The cross channel interface 202, and any wall of a flow chamber to be patterned, can be patterned with hot-embossing. Hot-embossing can be accomplished as a two step molding process. First, a silicon mold is fabricated using photolithography and reactive ion etching. The first step of the process generates a positive of the selected pattern. Next, a negative is created from the positive. The negative is formed by electroforming nickel to the positive mold. The electroforming is accomplished by applying a voltage difference between a nickel source and the silicon mold. This causes the nickel to flow into the silicon mold. In some implementations, prior to embossing, the patterned face of the nickel mold is soaked in a 1 mM solution of hexadecanethiol (HDT), which forms a self-assembled monolayer (SAM) to decrease surface energy to aid in subsequent polymer release.

The second step of the hot-embossing method includes placing the cross channel interface 202 in contact with the topographically patterned face of the nickel mold. The mold and cross channel interface 202 are sandwiched between two Kapton polyimide films and silicone rubber sheets to decrease sticking and add compliance. The stack is then placed in a uniformly heated, temperature- and pressure-controlled automatic hydraulic press. A light load is applied to the stack while the temperature is set to about 150° C. The load is applied for a specified dwell time before being cooled to 60° C. under constant pressure. Upon cooling, the newly patterned membrane is released from the nickel mold and analyzed for changes in pore size and geometry. The combination of the dwell time, pressure and temperature are selected such that the topographical pattern is fully created in the cross channel interface 202, but does not cause polymer material to flow into the pores 207. In some implementations, the dwell time was selected to be between about 10 and about 20 minutes, under a pressure of between about 700 kPa and about 850 kPa, and at a temperature of about 125° C. to about 175° C. For example, a dwell time of 15 minutes at 820 kPa and 150° C. preserves pore architecture. In other implementations, alternative embossing parameters and processes may be employed.

In some implementations, the method 500 of manufacturing a cell culture support device includes coupling the cross channel interface between the first and second polymer layers (step 506). As discussed above, in some implementations, the cross channel interface 202 is a component of the first or second polymer layers, and therefore, in these implementations, the first polymer layer 203 would be directly completed to the second polymer layer 201. In other implementations, the cross channel interface 202 is coupled between the first polymer layer and the second polymer layer. In certain implementations, the components of the cell culture support device 101 are reversibly coupled to one another. For example, a clamp can be used to couple the components together during an experiment and allow for the cross channel interface 202 to be removed after an experiment for further analysis.

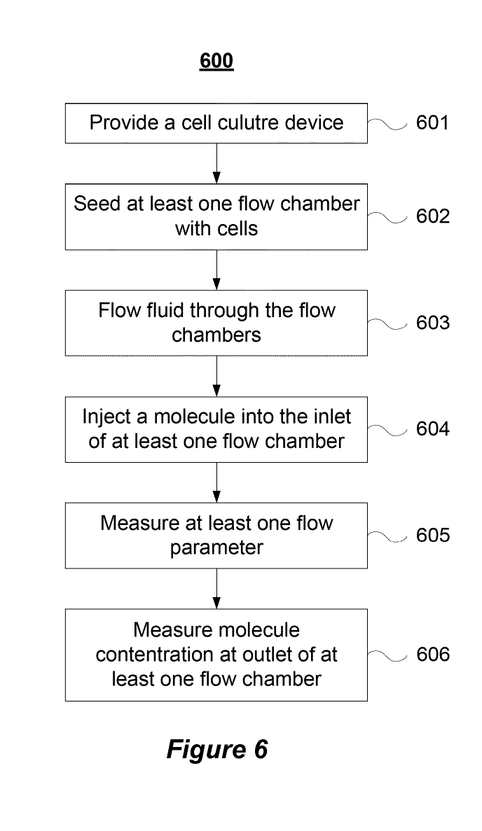

FIG. 6 is a flow chart of a method 600 for using a cell culture support device in a system similar to the system 100. The method 600 begins with the provisioning of a cell culture support device (step 601). Then, cells are seeded into at least one flow chamber of the cell culture support device (step 602). The method 600 continues with the flowing of fluid through the flow chambers of the cell culture support device (step 603) and the injection of a molecule into the inlet of at least one of the flow chambers (step 604). Responsive to flowing fluid through the flow chambers, at least one flow parameter is measured (step 605) and the concentration of the injected molecule is measured at an outlet of at least one flow chamber (step 606).

As set forth above, the method 600 begins by providing a cell culture support device (step 601), such as the cell culture support device 101 of FIG. 2. Next, cells are seeded into the cell culture support device (step 602). In some implementations, the cell culture support device 101 is provided pre-assembled, and in other implementations, the cell culture support device 101 is provided unassembled. For example, cells can be seeded onto the cross channel interface 202 prior to assembly of the cell culture support device 101. The cell seeded cross channel interface 202 can then be cultured in an incubator until the cells reach a maturity level appropriate for experimentation. In other implementations, cells are injected into the cell culture support device 101 with a syringe and allowed to adhere to the cross channel interface 202 and/or other surfaces of the flow chambers. In certain implementations, the cell culture support device 101 is sterilized prior to cellular seeding. For example, the cell culture support device 101 can be sterilized with ethylene oxide and then rinsed with 70% ethanol. Also prior to cellular seeding, in certain implementations, the cross channel interface 202 and/or remaining components of the cell culture support device 101 are coated with an agent, such as an agent to inhibit or encourage cellular growth. For example, surfaces of the cell culture support device 101 exposed to cells can be coated with an extracellular matrix, collagen IV, collagen I, laminin, fibronectin, agrin, nephrin, or similar proteins, Arginine-Glycine-Aspartic acid or similar peptides, or adhesive motifs.

The method 600 continues with the flowing of fluid through the cell culture support device (step 603). As described above in relation to FIG. 1, fluid flow through the chambers of the cell culture support device 101 is controlled with a pump 103. In some implementations, fluid is only flowed through one of the flow chambers. For example, a first chamber can act as a cellular well without perfusion, such that the cells within the camber are not exposed to shear stress caused by flowing fluid. In this example, nutrients or other agents can be transported to the cellular well through the pores 207 in a cross channel interface 202 that separate the cellular well from flowing fluid in a flow chamber beneath the cellular well.

The method 600 also includes injecting a molecule into the inlet of at least one flow chamber (step 604). The molecule can be a cell culture medium, cell nutrient, reagent, test agent, buffer fluid, reactant fluid, fixing agent, and/or stain. In some implementations, the injection of the molecule and/or the pump 103 is computer controlled so that a specific flow rate and molecule concentration is achieved within the cell culture support device 101. Example molecules to be injected can include, but are not limited to, water, sodium, potassium, chlorine and other ions; urea creatinine, and other metabolic products; oxygen, carbon dioxide, nitrogen, and other gases; macromolecules of defined molecular weights such as inulin, ficoll, dextran, albumin and other proteins; pharmaceutical agents and their metabolically-modified forms; toxins; cells and subcellular biological components such as platelets and microparticles; large particles of solids include micro and nano particles; lipid and other vesicles either synthetic or naturally-derived; bubbles or other gas-phase particles.

Responsive to flowing fluid through the cell culture support device, at least one flow parameter is measured (step 605). The measurement of the flow parameter can include parameters that are either directly measured, such as fluid flow rate and fluid pressure, derived measurements, such as shear stress measurements. In certain implementations, the measurements are made at the inlet, outlet and/or within the cell culture support device. In other implementations, cross channel permeability is measured. For example, hydraulic permeability, which measures the flux of a chemical, molecule or agent through a membrane at a given transmembrane pressure, can measured. In certain implementations, the transmembrane pressure is measured by direct measurements or by deriving the measurement based on pressure levels at channel inputs and outputs. The fluid flow rate can be quantified by measuring filling of a vessel of known size, measuring mass of inputs/outputs over time, flow visualization techniques, particle image velocimetry, and techniques using tracer elements or contrast agents.

Additionally, the method 600 includes measuring the concentration of the injected molecule at an outlet of at least one of the flow chambers (step 606). In some implementations, transport through the cross channel interface 202 (and in some implementations, the layer of cells seeded on the cross channel interface 202) is measured by injecting a molecule into an inlet of a first flow channel and then measuring the concentration of the molecule at the outlet of a second flow channel.

In some implementations, the transport of specific species across the membrane is analyzed by evaluating the concentrations of solutes, particles and other components of fluids in a cellular flow chamber, at the inlet of a cellular flow chamber, and/or at the outlet of a cellular flow chamber. In certain implementations, the concentration of the molecules is measured with a sensor, a molecule selective dye, a soluble nanosensor, a molecular label, such as a radioactive label or tracer. In some implementations, the evaluation of concentration and flow can be configured such that a sieving coefficient of a molecule or component is quantified for the device, cross channel interface 202, and/or the membrane-cell construct. The sieving coefficient is the concentration of a specific analyte in the fluid passing through the membrane divided by the concentration of that same analyte in the fluid being fed to the membrane. The sieving coefficient can reflect the selectivity of a porous membrane.

### EXAMPLES

### I. Topographic Pattering

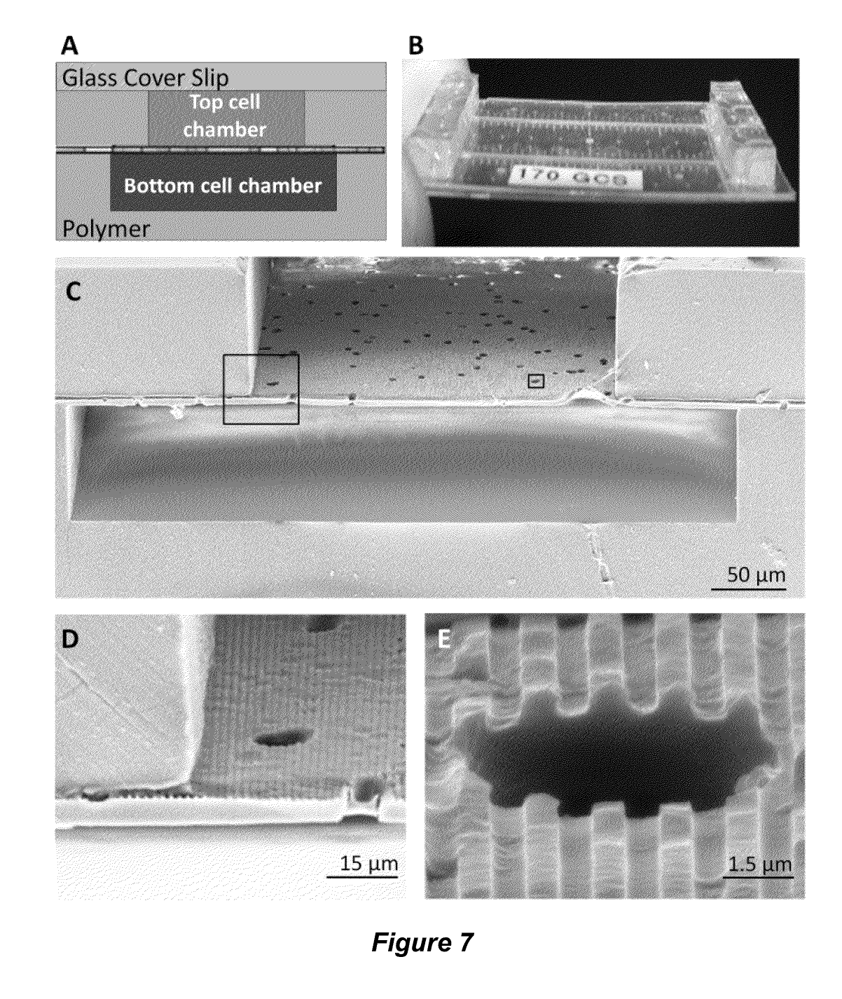

FIG. 7A-7E illustrates a series of images, at different magnification, of a cell culture support device manufactured using the above described hot-embossing method. In this example, the cross channel interface was manufactured as an independent topographically-patterned membrane assembled into a cell culture support device. FIG. 7A is a schematic of the overall cross-sectional architecture of the cell culture support device. As in the cell culture support device 101 of FIG. 2, the schematic illustrates a top cell chamber defined in a first polymer layer and a bottom cell chamber defined in a second polymer layer. A cross channel interface is coupled between the first polymer layer and second polymer layer, and a cover slide provides the roof for the top cell chamber. FIG. 7B illustrates an assembled cell culture support device, of which FIGS. 7C-7E provide greater detail.

FIG. 7C is a scanning electron micrograph illustrating the cross section of the device in FIG. 7B. The micrograph shows the porous nature of the cross channel interface separating the top and bottom chambers. FIGS. 7D and 7E are scanning electron micrographs magnifying the inserts of FIG. 7C. FIGS. 7D and 7E illustrate the well-defined groove topography coexisting with the porous architecture.

### II. Topographic Examples

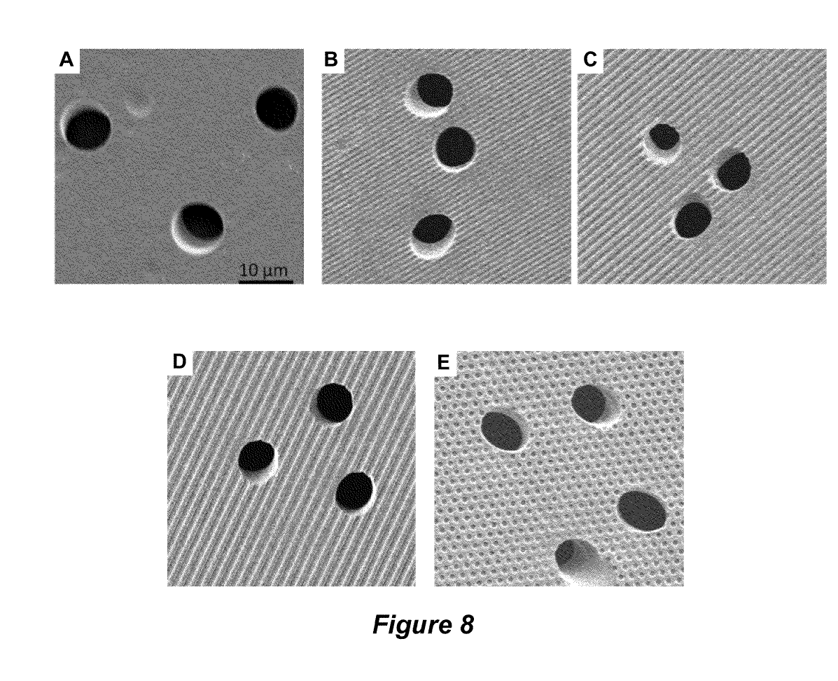

FIGS. 8A-E are a series of scanning electron micrographs showing example pattern topographies created with the hot-embossing method described above on cross channel interfaces with 8 μm diameter pores. FIG. 8A is an image of a cross channel interface 202 including three pores and a smooth topography. FIGS. 8B-8D illustrate the ridge and groove topography discussed in relation to FIGS. 4A-4C. FIGS. 8B-8D also illustrate the effect of ridge width on topography. The width of the ridges in FIG. 8B is 0.5 μm, 0.75 μm in FIG. 8C, and 1.0 μm FIG. 8D. Similarly, FIG. 8E illustrates a cross channel interface with evenly spaced 1.0 μm pits. In FIGS. 8B-8E, the topographical features are 0.75 μm deep.

### III. Hot-Embossing Parameters

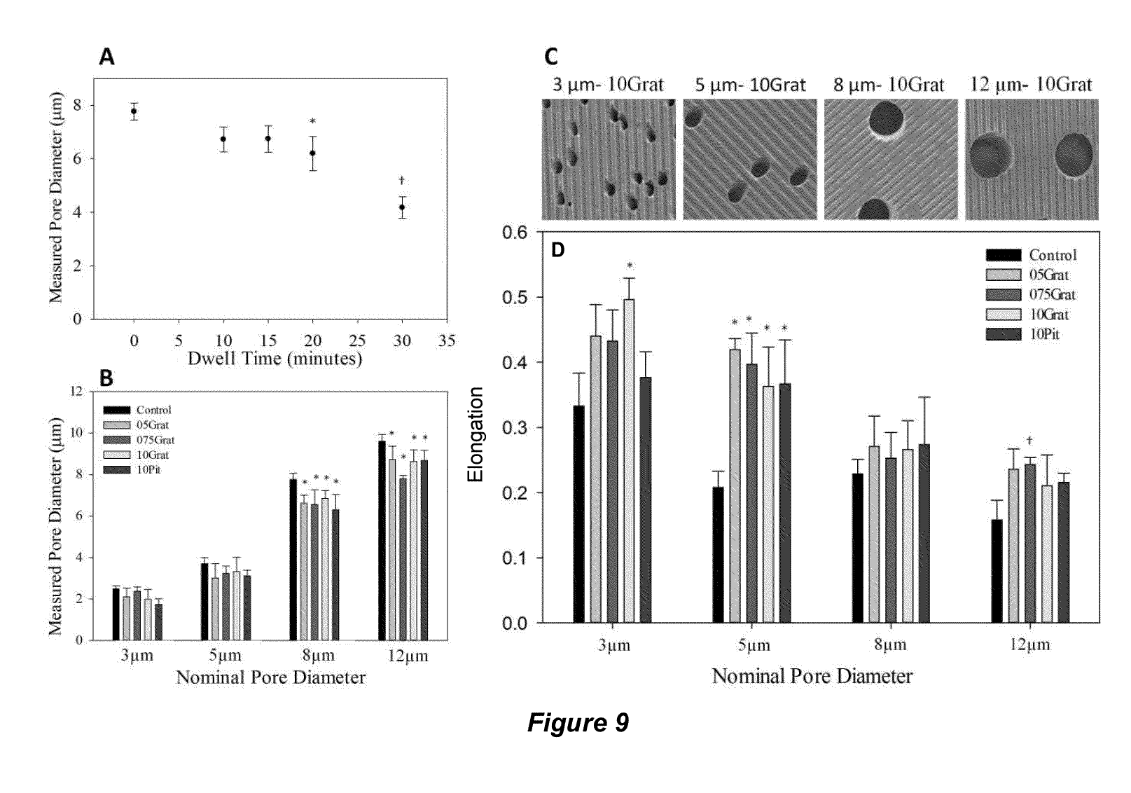

As discussed above, the embossing of the topographical pattern onto the cross channel interface are done in a controlled manner as to not alter the pore architecture. To determine the appropriate embossing parameters, a series of cross channel interfaces were hot-embossed under a constant load of 820 kPa at 150° C. For the trials the dwell time ranged from 10 to 30 minutes. FIG. 9A is a graph illustrating how pore diameter (y axis) changes with dwell time (x axis). The plot in FIG. 9A shows that nominal pore size did not significantly change for dwell times less than 15 minutes when compared to the pre-embossed cross channel interface. However, as dwell time increased past 20 minutes, pore diameter significantly decreased when compared to pre-embossing diameters.

FIG. 9B is a series of bar charts that illustrate how different topographies affect pore diameter during the embossing process. For this experiment, cross channel interfaces were created to have 3 μm, 5 μm, 8 μm, or 12 μm diameter pores. The diameter of the pores on each cross channel interface were measured and averaged to serve as controls for the experiment. The cross channel interfaces were then hot-embossed with a dwell time of 20 minutes under 820 kPa at 150° C. The resulting pore diameters were measured and averaged.

FIG. 9B shows that embossing reduces pore diameter for membranes with large pores, such as the 8 μm and the 12 μm diameter pores, but does not reduce pore diameter for pores with small pore diameters, such as the 3 μm and the 5 μm diameter pores. The 8 μm and 12 μm pore cross channel interfaces showed a decrease in pore diameter over all topographies after embossing. Also, while a pore diameter of 12 μm was desired for the fourth group (the 12 μm group), the actual pore diameter was measured to be closer to 10 μm. Thus, the decrease in pore diameter caused by the hot-embossing process in the 12 μm cross channel interface, although statistically significant, was smaller than the difference between the desired control pore diameter and the actual measured 12 μm pore diameter.

Some decrease in pore diameter is expected after embossing due to the flow of the polymer under high temperature and pressure. Pore deformation was independent of pattern type. A dwell time of 10-15 minutes provided a good balance of pattern transfer onto the cross channel interfaces without significantly changing pore diameter.

In some experiments, pores were not perfect circles and the elongation of the pores was exacerbated during the hot-embossing process. FIG. 9C is a series of scanning electron micrographs illustrating the differences in elongation of 3-12 μm pores embossed with a ridge and groove topography (a 10 Grat topography corresponds to 1.0 μm wide ridges spaced 2.0 μm apart). FIG. 9D is a bar chart, with the same groupings represented in FIG. 9B, and illustrates the amount of elongation, represented as a fraction, induced by hot-embossing with a dwell time of 20 minutes under 820 kPa at 150° C. The average pore in the non-embossed control cross channel interfaces exhibited a slightly elongated shape with a fraction of elongation ranging from 0.16 for 12 μm pores to 0.34 for 3 μm pores. FIG. 9D shows how the fraction of elongation for pores after embossing was dependent on initial pore diameter and in some cases, the topography. Cross channel interfaces with smaller pore diameters, i.e. 3 μm and 5 μm, yielded significantly higher fractions of pore elongation when compared to larger pore sizes. Cross channel interfaces with a pore size of 3 μm exhibited pores with an average elongation fraction of almost 0.5 when embossed with the 10 Grat pattern, a 49% increase from the control. On average, elongation of the 3 μm pores increased by 37% across all linear patterns. For larger pore sizes, i.e. 8 μm and 12 μm, the change in pore elongation became insignificant across most topographies when compared to the control pore geometry. The elongation of 8 μm pores increased by an average of only 15.3% among linear patterns. Cross channel interfaces with 12 μm pores had the lowest fraction of elongation, with an average elongation of 0.23 among linear patterns. Topographical features in the form of 1 μm pits had the least affect on pore elongation for small and large pore sized membranes, with a range from 0.37 for 3 μm pores to 0.22 for 12 μm pores.

### IV. Cells Proliferate on and Respond to Porous Membrane Topography

A cell culture support device, similar to that of the device in FIGS. 7A-7E, consisting of two channels separated by the patterned porous cross channel interface (also referred to as a membrane) served as a platform to characterize the response of renal epithelial cells cultured on the patterned porous membrane. The device's performance was evaluated in three ways. First, HK-2 response to unpatterned and patterned membranes outside the device was characterized by analyzing cellular alignment. Second, the cell culture support device was evaluated by scanning electron microscopy and optical microscopy to verify channel geometry and alignment. Third, immunofluorescent techniques were used to label markers indicative of a reabsorptive epithelial layer for cells cultured in the cell culture support device.

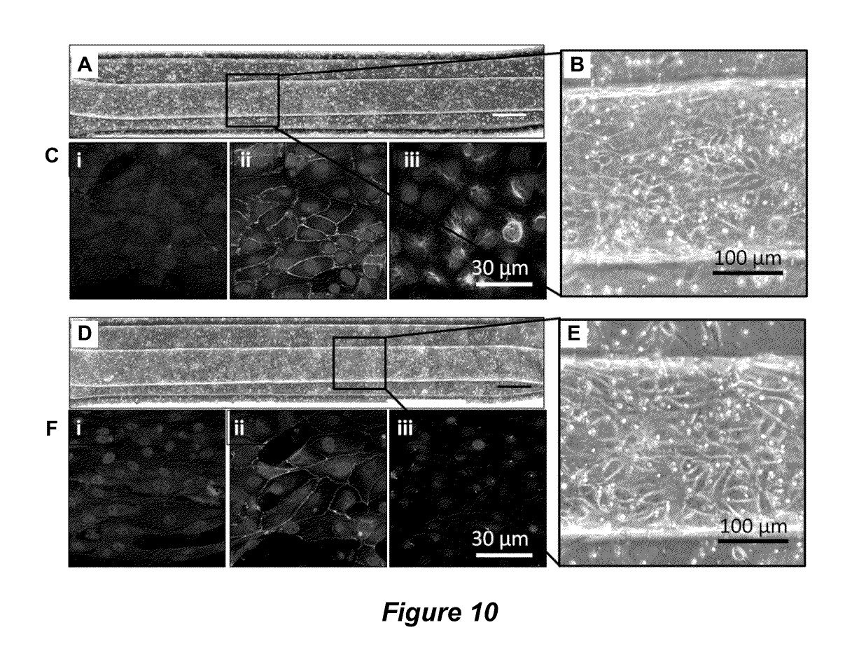

During the experiments, HK-2 cells and renal proximal tubule epithelial cells (RPTECs) proliferated from initial seeding to confluency within the cell culture support device over approximately 4 days. A uniform initial seeding density and appropriate culture time yielded complete confluency of both HK-2 and RPTECs over the cell culture support device channel area of 1.25 mm² shown respectively in the brightfield composites in FIG. 10A and FIG. 10D, with higher magnification views provided in FIGS. 10B and 10F. HK-2 and RPTEC monolayers expressed paxillin (FIGS. 10C(i) and 10F(i)), a typical epithelial marker of focal adhesions; ZO-tight junction complexes (FIGS. 10C(ii) and 10F(ii)); and acetylated tubulin, an indicator of primary cilia and cytoplasmic microtubules (FIGS. 10C(iii) and 10F(iii)). Paxillin expression in HK-2 samples signified focal adhesions that were less discrete with a weaker signal than RPTEC samples. Both HK-2s and RPTECs expressed ZO-1 in distinct borders outlining the perimeter of cells, indicating initial formation of a tight-junction-based sealed epithelial barrier. Acetylated tubulin morphology differed between the HK-2s and RPTECs. The HK-2s showed somewhat distributed cytoplasmic microtubules, but distinct primary cilia were not expressed on the apical surface. The RPTECs expressed acetlylated tubulin in a single punctuate spot on the apical surface of each cell, indicating formation of a primary cilia.

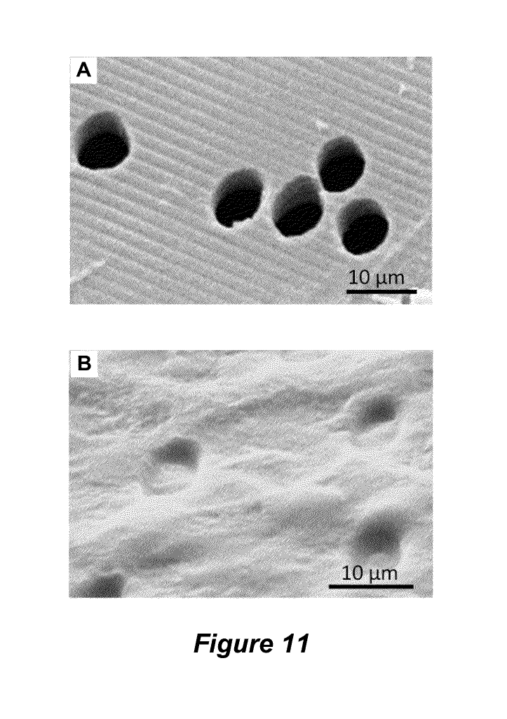

Formation of the complete, confluent cellular monolayer within the channel layer allows interrogation of the layer for permeability, a requisite for a reabsorptive barrier. As the layer is confluent, the cell culture support device allows fluidic and/or electrical access to any point on the flow chamber and thus the direct measurement of cellular transport. Therefore, the cell culture support device allows for the quantification of reabsorptive properties. Formation of ZO-1 junctions indicate an epithelial barrier capable of active transport. The cellular junctions can be improved by conditioning the cells with mechanical and/or other stimuli. The HK-2 cells formed a more mature monolayer due to its longer culture time, causing subtle differences in paxillin expression. Focal adhesions in highly developed monolayers are not discrete and tend to have a weaker signal, which was seen in the HK-2 samples as compared to the RPTECs. The lack of primary cilia in HK-2 cells was not abnormal. Primary cilia may not be fully expressed in HK-2 populations if their formation is not enhanced by serum starvation or shear stress. Cytoplasmic tubulin was more prevalent compared with RPTEC, with signs of a microtubule-organizing center that may nucleate cilia development. The presence of primary cilia in the RPTECs indicated the cells will be responsive to mechanical stimuli, such as shear stress, as the cilia can serve to transduce mechanical signals to chemical activity. Continuous flow of nutrient rich fluid through the cell culture support device delivers nutrients to the cell populations, thereby creating more favorable conditions for long term cell culture in a small channel volume while simultaneously mimicking the filtrate flow seen by proximal tubule cells in vivo. Finally, as shown by the scanning electron micrographs of FIGS. 11A and 11B, the cells block the pores of the membrane. FIG. 11A is a scanning electron micrograph of a membrane prior to cellular seeding and FIG. 11B is a membrane showing that the seeded cells block the pores of the membrane. FIG. 11B indicates transport across the membrane-cell layer construct can be limited to transcellular transport if paracellular transport is limited through tight junction and other cell-cell junction formation. With the ability to stimulate cells mechanically; interrogate cells with imaging, electrical, and fluidic means; and support growth of an epithelial layer expressing indications of a mechanically-responsive reabsorptive barrier, the patterned porous membrane of the cell culture support device allows the quantification reabsorptive barrier function.

What is claimed:

1. A cell culture support device, the cell culture support device comprising:
a first polymer layer with a first flow chamber defined therethrough;
a second polymer layer with a second flow chamber defined therethrough; and
a surface between the first polymer layer and the second polymer layer, and separating the first flow chamber from the second flow chamber, wherein the surface further includes:
a plurality of pores configured to allow communication and transport between the first flow chamber and the second flow chamber, and
a first pattern formed on at least one face of the surface, wherein the first pattern is independent of the geometry of the plurality of pores and the first pattern preserves the plurality of pores.

2. The device of claim 1, wherein the surface is a membrane.

3. The device of claim 1, wherein at least one of the first flow chamber and the second flow chamber is a cell chamber.

4. The device of claim 1, wherein a top layer is coupled to the first polymer layer and configured to allow imaging of the surface.

5. The device of claim 1, wherein the first pattern is one of a topographic pattern and a chemical pattern.

6. The device of claim 1, wherein the first pattern is selected for growing a first type of cells thereon.

7. The device of claim 1, wherein a second pattern is formed on at least one face of the surface.

8. The device of claim 7, wherein the second pattern is selected for growing a second type of cells thereon.

9. The device of claim 1, wherein the first pattern is selected to alter the geometry of the pores.

10. The device of claim 1, wherein the configuration of the plurality of pores is selected to produce a specific type of interaction between the first flow chamber and the second flow chamber.

11. The device of claim 1, wherein the size of the plurality of pores is selected to prevent cell migration between the first and second flow chambers and to allow cell nutrients and cell signaling analytes to migrate between the first and second flow chambers.

12. The device of claim 11, wherein the size of the plurality of pores is between about 3 μm and about 15 μm.

13. The device of claim 1, wherein the first pattern is selected to elicit a particular arrangement, function, shape, alignment, or density of cellular growth.

14. The device of claim 1, wherein geometry of the plurality of pores is selected to elicit a particular arrangement, function, shape, alignment, or density of cellular growth.

15. The device of claim 1, wherein at least one of the first and second polymer layers comprise a biodegradable polymer.

16. The device of claim 1, wherein the surface comprises a biodegradable polymer.

17. The device of claim 1, wherein the first pattern is selected to influence a degradation rate of the surface.

18. The device of claim 1, wherein the first pattern is selected to facilitate cell attachment to particular locations of the surface.

19. A method for fabricating a cell culture support device, the method comprising:
forming a first flow chamber in a first polymer layer,
forming a second flow chamber in a second polymer layer;
forming a plurality of pores through a surface, wherein the plurality of pores have a specific size;
selecting a first pattern for at least one face of the surface, wherein the selection of the first pattern is independent from the selection of the pore size;
forming the selected pattern on the at least one face of the surface, wherein the formation of the selected pattern preserves the plurality of pores through the surface; and
coupling the surface to the first polymer layer and the second polymer layer such that the surface separates the first flow chamber from the second flow chamber.

20. The method of claim 19, further comprising seeding cells into at least one of the first flow chamber and second flow chamber.

21. The method of claim 19, wherein the surface is a membrane.

22. The method of claim 19, wherein the size of the plurality of pores is between about 3 μm and about 15 μm.

23. The method of claim 19, wherein the plurality of pores have a specific pore density.

24. The method of claim 19, wherein the first pattern is one of a topographic pattern and a chemical pattern.

25. The method of claim 19, wherein the selection of the first pattern is based on a type of cell to be grown on the surface.

26. The method of claim 19, further comprising:
selecting a second pattern for at least one face of the surface, wherein the selection of the second pattern is independent from the selection of the pore size; and
forming the selected second pattern on the at least one face of the surface, wherein the formation of the selected second pattern preserves the plurality of pores through the surface.

27. The method of claim 26, wherein the first pattern is different from the second pattern.

28. The method of claim 19, wherein the first pattern is selected to elicit a particular arrangement, function, shape, or density of cells grown on the surface.

29. The method of claim 19, wherein at least one of the first and second polymer layers comprise a biodegradable polymer.

30. The method of claim 19, wherein the surface comprises a biodegradable polymer.

31. The method of claim 19, wherein the first pattern is selected to influence a degradation rate of the surface.

32. The method of claim 19, further comprising:
selecting the first pattern to facilitate cellular attachment to particular locations of the surface; and
selecting the location for the plurality of pores such that the plurality of pores align with the locations of the cellular attachment.

33. A cell culture support system, the system comprising:
a first polymer layer, with a first flow chamber defined therethrough;
a second polymer layer, with a second flow chamber defined therethrough;
a surface, wherein the surface includes a plurality of pores configured to allow communication and transport between the first flow chamber and the second flow chamber and a first pattern formed on at least one face of the surface, wherein the first pattern is independent of a geometry of the plurality of pores, and the first pattern preserves the plurality of pores; and
an imager configured to image a face of the surface.

34. The system of claim 33, further comprising a means for coupling the surface between the first and second polymer layers such that the surface separates the first flow chamber from the second flow chamber.

35. The system of claim 33, further comprising a flow meter configured to measure flow through at least one of the first flow chamber and second flow chamber.

36. The system of claim 33, further comprising a pressure sensor configured to measure the pressure at an inlet and outlet of at least one of the first flow chamber and second flow chamber.

37. The system of claim 33, further comprising a fluid pump configured to flow fluid through at least one of the first flow chamber and the second fluid chamber.

38. The system of claim 33, further comprising a means for injecting a macro-molecule into an inlet of at least one of the first flow chamber and second flow chamber, and a means for collecting fluid from an outlet of at least one of the first flow chamber and second flow chamber.

39. The system of claim 33, wherein the imager is a microscope.
